(19) United States
(12) Patent Application Publication
(10) Pub. No.: US 2026/0207940 A1
(43) Pub. Date: Jul. 23, 2026

(54) A MINIATURIZED WIRELESS MULTI-CHIP MODULE FOR TREATMENT OF AN EAR/BRAIN/SYSTEMIC DISORDER

(71) Applicant: THE REGENTS OF THE UNIVERSITY OF CALIFORNIA, Oakland, CA (US)

(72) Inventors: Michael GREEN, Orange, CA (US); Hamid DJALILIAN, Irvine, CA (US)

(73) Assignee: THE REGENTS OF THE UNIVERSITY OF CALIFORNIA, Oakland, CA (US)

(21) Appl. No.: 19/139,159

(22) PCT Filed: Dec. 14, 2023
(86) PCT No.: PCT/US2023/084034

§ 371 Date: Jun. 13, 2025

Related U.S. Application Data
(60) Provisional application No. 63/432,518, filed on Dec. 14, 2022.

(51) Int. Cl. A61N 1/36

(52) U.S. Cl. CPC A61N 1/36036; A61N 1/361; A61N 1/36132; A61N 1/36135

(57) ABSTRACT

A system for stimulating structures outside, behind and proximate to a subject's eardrum is provided. The system includes a multi-chip module that is powered and controlled wirelessly. The multi-chip module has a size such that it is implantable in a subject's external ear, middle ear, and/or inner ear. Characteristically, the multi-chip module is configured to minimally-invasively electrically stimulate a structure in the subject's middle or inner ear. A transmitter is configured to wirelessly transmit control signals and power to the multi-chip module.

### CROSS-REFERENCE TO RELATED APPLICATIONS

This application claims the benefit of U.S. provisional application Ser. No. 63/432,518 filed Dec. 14, 2022, the disclosure of which is hereby incorporated in its entirety by reference herein.

### TECHNICAL FIELD

In at least one aspect, the present invention relates to devices for stimulating middle or inner ear structures.

### BACKGROUND

Tinnitus, which is the undesirable perception of sound in the absence of an external source, affects 10-15% of the population but is known to be difficult to treat. A recent advance in the treatment of tinnitus is to electrically stimulate the inner ear tissue with well-defined current signals of adjustable frequencies, amplitudes, and waveshapes while maintaining a balanced charge to avoid damage to the inner ear. The amplitude of the stimulation current should be able to reach at least 1.25 mA in order to effectively treat tinnitus.

The major challenge posed by this treatment is that the available commercial equipment used to generate the stimulus is very bulky and normally available only inside hospitals; moreover, the stimulus cannot be easily implanted inside the inner ear.

Accordingly, there is a need for improved devices for treating disorders related to structures behind the eardrum.

### SUMMARY

In at least one aspect, a system for stimulating structures behind and proximate to a subject's ear is provided. The system includes a multi-chip module that is powered and controlled wirelessly. Characteristically, the multi-chip module has a size such that it is implantable in a subject's middle or inner ear. Advantageously, the multi-chip module is configured to minimally-invasively electrically stimulate a structure in a subject's external ear, middle ear, and/or inner ear. The system can further include a transmitter configured to wirelessly or by a connection to a wire transmit control signals and power to the multi-chip module.

In another aspect, an integrated circuit used for tinnitus treatment is provided. This chip, realized using TSMC 0.18 μm BCD high-voltage process, is capable of generating an arbitrary current stimulus into the inner ear without the need for an FPGA or any other off-chip control circuitry. Used as part of a multi-chip module, this core chip contains an 8-bit digital-to-analog converter, an amplitude control block, a charge-balanced H-bridge stimulator, an SRAM, a ROM, a serial-to-parallel converter, and a central control unit. The chip also supports a charge balance mode with a programmable duty cycle.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIGS. 1A and 1B. (A) Illustration of the implantable module. (B) Simplified inner-ear model.

FIG. 2. Multi-chip module configuration

FIG. 3. Block diagram.

FIG. 4. Stimulus waveforms showing charge balance.

FIG. 5. Digital to analog converter.

FIG. 6. Amplitude control.

FIG. 7. High-voltage drive & charge balance.

FIG. 8. Level shifter.

FIG. 9. Chip layout.

FIG. 10. Test bench.

FIGS. 11A, 11B, 11C, and 11D. Simulation results: (a) enable signal; (b) control_5 and control_15 bias voltages; (c) differential current stimulus delivered to load; (d) differential voltage across load.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numerical quantity, in a refinement, the term “less than” includes a lower non-included limit that is 5 percent of the number indicated after “less than.” A lower non-included limit means that the numerical quantity being described is greater than the value indicated as a lower non-included limit. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, 1 percent, or 0 percent of the number indicated after “less than.”

For any device described herein, linear dimensions and angles can be constructed with plus or minus 50 percent of the values indicated rounded to or truncated to two significant figures of the value provided in the examples. In a refinement, linear dimensions and angles can be constructed with plus or minus 30 percent of the values indicated rounded to or truncated to two significant figures of the value provided in the examples. In another refinement, linear dimensions and angles can be constructed with plus or minus 10 percent of the values indicated rounded to or truncated to two significant figures of the value provided in the examples.

With respect to electrical devices, the term “connected to” means that the electrical components referred to as connected to are in electrical communication. In a refinement, “connected to” means that the electrical components referred to as connected to are directly wired to each other. In another refinement, “connected to” means that the electrical components communicate wirelessly or by a combination of wired and wirelessly connected components. In another refinement, “connected to” means that one or more additional electrical components are interposed between the electrical components referred to as connected to with an electrical signal from an originating component being processed (e.g., filtered, amplified, modulated, rectified, attenuated, summed, subtracted, etc.) before being received to the component connected thereto.

The term “electrical communication” means that an electrical signal is either directly or indirectly sent from an originating electronic device to a receiving electrical device. Indirect electrical communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electrical communication can be accomplished with wired components, wirelessly connected components, or a combination thereof.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10% of the value or relative characteristic.

The term “electrical signal” refers to the electrical output from an electronic device or the electrical input to an electronic device. The electrical signal is characterized by voltage and/or current. The electrical signal can be stationary with respect to time (e.g., a DC signal) or it can vary with respect to time.

The term “electronic component” refers is any physical entity in an electronic device or system used to affect electron states, electron flow, or the electric fields associated with the electrons. Examples of electronic components include, but are not limited to, capacitors, inductors, resistors, thyristors, diodes, transistors, etc. Electronic components can be passive or active.

The term “electronic device” or “system” refers to a physical entity formed from one or more electronic components to perform a predetermined function on an electrical signal.

It should be appreciated that in any figures for electronic devices, a series of electronic components connected by lines (e.g., wires) indicates that such electronic components are in electrical communication with each other. Moreover, when lines directed connect one electronic component to another, these electronic components can be connected to each other as defined above.

Throughout this application, where publications are referenced, the disclosures of these publications in their entireties are hereby incorporated by reference into this application to more fully describe the state of the art to which this invention pertains.

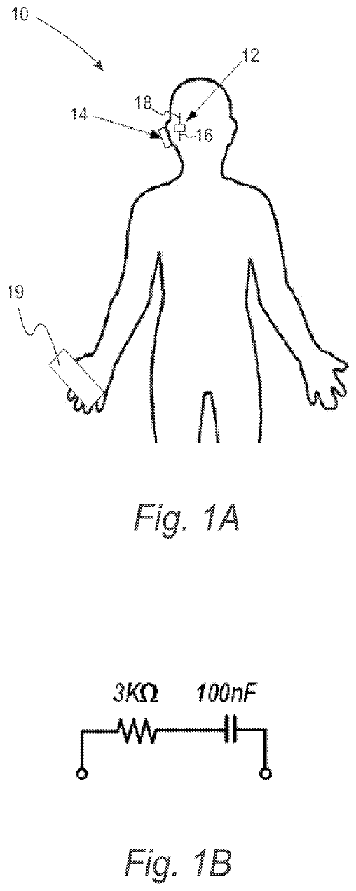

Referring to FIGS. 1A and 1B, schematics of a system for stimulating structures behind and proximate to a subject's ear is provided. FIG. 1A illustrated the system implanted in a subject while FIG. 1B provides a schematic of an equivalent circuit. System 10 is a fully implantable multi-chip module that can deliver a well-defined charge-balanced current stimulus to the inner-ear tissue. As shown in FIG. 1(A), the multi-chip module needs to be fully miniaturized such that it can be implanted inside the inner ear. The transmitter, also illustrated in FIG. 1(A), is a separate device that resides outside the ear and thus does not need to be implantable; since its form factor is not critical, it can be realized using commercially available chips. The energy powering the chips is received wirelessly through an implantable receiving coil; at the same time, the data encoding the waveshape, frequency, and magnitude of the current stimulus is modulated onto the same coil that receives the energy. While modeling the impedance of the inner ear can be complex, an adequate model suitable for circuit simulation is the series connection of a resistor and capacitor as shown in FIG. 1(B). The maximum impedance magnitude of this model at the lowest usable stimulation audio frequency of 150 Hz-approximately 11 kΩ—will require a 20V power supply to deliver the required current amplitude of 1.25 mA while providing some extra voltage headroom to ensure performance across PVT. Thus an advanced high-voltage bipolar-CMOS-DMOS (BCD) process is the best choice for this application.

Referring to FIGS. 1A and 1B, system 10 includes a multi-chip module 12 that is powered and controlled wirelessly. Multi-chip module 12 has a size such that it is implantable in a subject's middle or inner ear. Advantageously, the multi-chip module 12 is configured to minimally-invasively electrically stimulate a structure in a subject's external ear, middle ear, and/or inner ear (e.g., a structure behind and proximate to a subject's eardrum). Transmitter 14 is configured to wirelessly transmit control signals and power to the multi-chip module. Alternatively, transmitter 14 is configured to transmit control signals and power to the multi-chip module via a connected wire. In a refinement, the implanted device (e.g., multi-chip module 12) and non-implanted device (e.g., transmitter 14) have unidirectional or bidirectional communication. Characteristically, the multi-chip module includes a first electrode 16 configured to contact the structure in the subject's external ear, middle ear, and/or inner ear and a minimum of one other electrode 18 configured to contact another proximate structure to operate as ground.

In another aspect, the user has a handheld controller 19 that allows changing parameters. In a refinement, a device (e.g., handheld controller 19) applies artificial intelligence to change stimulation parameters based on physiological and non-physiological measurements.

In another aspect, the transmitter 14 is configured to transmit an encoded control signal having information about the waveform, the frequency, and the amplitude of a signal to be used for stimulating the structure in the subject's external ear, middle ear, and/or inner ear. Typically, the waveform is a periodic waveform (e.g., sine wave, square wave, triangle wave, and the like). In a refinement, the signal used to stimulate the structure in the subject's middle or inner ear has a voltage between 0 and 20 volts and/or a frequency from 20 Hz to 20 kHz and/or a current up to 1.25 mA. In a refinement, the signal used to stimulate the structure in the subject's middle or inner ear has a voltage between of at least in order of preference 0 volts, 0.1 volts, 0.5 volts, 1 volts, 2 volts, 3 volts, 5 volts, 7 volts, or 10 volts and at most 25 volts, 20 volts, 18 volts, 16 volts, 15 volts, or 14 volts. In a refinement, the signal used to stimulate the structure in the subject's middle or inner ear has a current of at least 0 mA, 0.1 mA, 0.2 mA, 0.3 mA, 0.4 mA, 0.5 mA, or 0.6 mA and at most 2, 1.8 mA, 1.5 mA, 1.3 mA, 1.25 mA, or 1 mA. In a refinement, the signal used to stimulate the structure in the subject's middle or inner ear has a frequency of at least 20 Hz, 100 Hz, 500 Hz, 1 kHz, 3 kHz, 5 kHz, or 10 kHz and at most 30 kHz, 20 kHz, 15 kHz, 10 kHz, 5 kHz, 3 kHz, or 1 kHz. Advantageously, the structure in the subject's external ear, middle ear, and/or inner ear can be a cranial nerve, a branch thereof, or a structure in close proximity to a cranial nerve.

In a variation, the multi-chip module is implanted in a subject to stimulate the facial nerve (cranial nerve VII) or one of its branches. In this variation, the multi-chip module can be used to treat facial paralysis, facial spasms, synkinesis, obesity, or weight loss.

In another variation, the multi-chip module is implanted in a subject to stimulate the vestibulocochlear nerve (cranial nerve VIII). In this variation, the multi-chip module is used to treat hearing loss, tinnitus, or balance issues.

In another variation, the multi-chip module is implanted in a subject to stimulate the glossopharyngeal nerve (cranial nerve IX). In this variation, the multi-chip module can be used to treat weight loss or to treat the subject with salivation disorders or dry mouth.

In another variation, the multi-chip module is implanted in a subject to stimulate the vagus nerve (cranial nerve X). In this variation, the multi-chip module is used to treat epilepsy, depression, anxiety, rheumatologic, metabolic, gastrointestinal, urologic, infectious, a neurological or other psychiatric disorder.

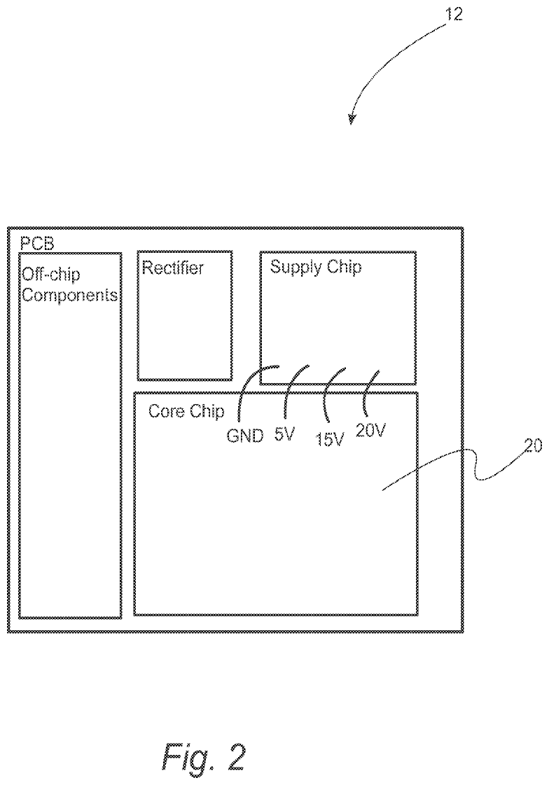

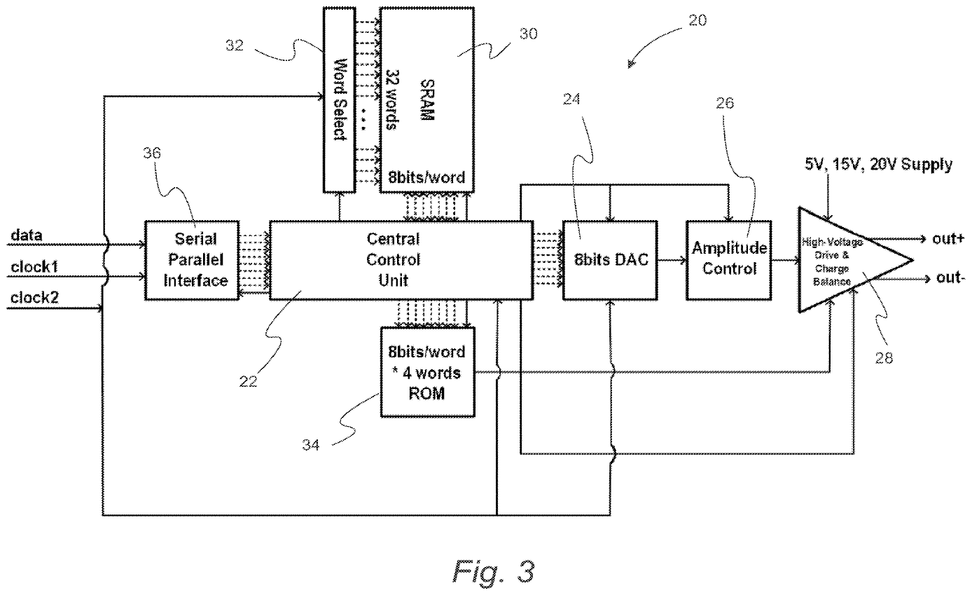

Referring to FIGS. 2 and 3, schematics of a multichip module are provided. The multi-chip module 12 includes a core chip 20 which includes a central control unit 22 and a digital-to-analog converter 24 in electrical communication with the central control unit. The digital-to-analog converter 24 receives a first set of control bits corresponding to binary-weighted analog currents and a second set of control bits that are decoded into control signals corresponding to thermometer-weighted analog currents. The digital-to-analog converter 24 outputs a first and second output current. Alternatively expressed, digital-to-analog converter 24 is in electrical communication with the central control unit, with the digital-to-analog converter output determined by a first set of control bits corresponding to binary-weighted analog currents and a second set of control bits that are decoded into control signals corresponding to thermometer-weighted analog currents, the digital-to-analog converter outputting a first and second output current. In a refinement, the digital-to-analog converter 24 is configured to operate with its full-scale amplitude. An amplitude control block 26 is driven by (i.e., is in electrical communication with) the digital-to-analog converter 24 is configured to receive the first and second output currents from the digital-to-analog converter and to generate differential current signals with tunable amplitudes. A charge-balanced H-bridge stimulator 28 in electrical communication with the amplitude control block. The charge-balanced H-bridge stimulator 28 is configured to receive the differential current signals with tunable amplitudes from the amplitude control block. The charge-balanced H-bridge stimulator 28 is also configured to operate in a first mode in which residual charge inside ear tissue is discharged and a second mode in at least one cranial nerve is stimulated.

In a variation, the core chip 12 further includes static random-access memory (SRAM) 30 is driven by (i.e., is in electrical communication with) the central control unit 22. The SRAM 30 is configured to store data that specifies signal parameters. In a refinement, the core chip 12 further includes word selection circuitry 32 in communication with the SRAM 30 and the central control unit 22. The core chip 12 further can also include read-only memory (ROM) 34 driven by (i.e., in electrical communication with) the central control unit 22. The ROM 34 is configured to store data that specifies signal parameters. In a further refinement, the core chip further includes a serial-to-parallel converter 36 configured to convert signal data into parallel data that is provided to the central control unit.

The following examples illustrate the various embodiments of the present invention. Those skilled in the art will recognize many variations that are within the spirit of the present invention and scope of the claims.

### System Description

An illustration of the multi-chip module is shown in FIG. 2, which shows a PCB on which is mounted the core chip, a dc-to-de voltage converter chip, a rectifier, and some off-chip components. The off-chip components consist primarily of capacitors that are used with the de-to-dc converter. The rectifier may be built with four discrete diodes. The entire module plus the implantable receiving coil must have dimensions no larger than about 4 mm on a side in order to be implantable into the patient's inner ear.

The core chip within the multi-chip module is shown in FIG. 2, whose system block diagram is shown in FIG. 3. The overall operation of the system is described as follows. A digital control sequence is modulated onto the ac signal that transfers energy between two coils used for wireless power transfer. Once the power transfer begins, a specific 12-bit code is transmitted that initiates a handshaking sequence with the receiver. Following that sequence, the actual data that specifies the signal parameters is transferred into the chip's SRAM and ROM as illustrated in FIG. 3. Finally another 12-bit code that specifies the end of transfer is sent, after which the circuit starts generating the current stimulus.

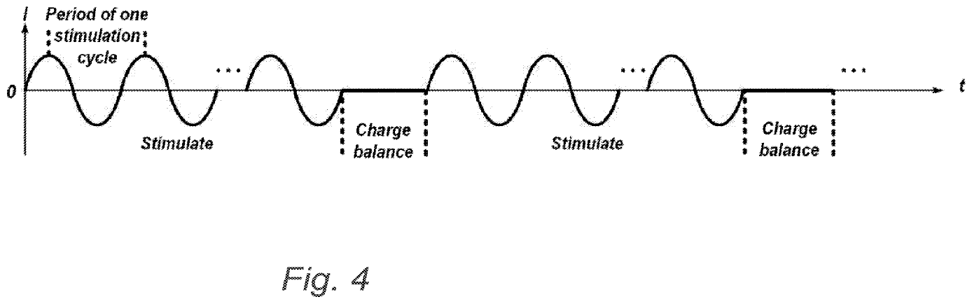

The analog circuit operation of the core chip is described as follows. On power up, the core chip is put into the charge-balanced state, where the two outputs that are connected to the inner ear are shorted, thus removing any residue charge. After the data has been transferred into the SRAM and the ROM through the data and clock1 wires shown in FIG. 3, the chip begins to generate the current stimulus as shown in FIG. 4. The number of consecutive stimulation cycles, the period of one stimulation cycle, and the duration of the charge balance time are all made adjustable: The number of consecutive cycles can be set between 7 and 2047; the period of one stimulation cycle can be set between 50 μs and 6.67 ms (corresponding to a frequency range of 150 Hz to 20 kHz for the stimulus waveform); and the duration of the charge-balance time can be set between 16 ms and 1.28 s. All of the above functions are set by the central control unit shown in FIG. 3.

### Core Circuit Description

The amplitude of the voltage swing across the ear tissue should be able to reach at least 14V during the stimulus mode while also periodically shorting the load during the charge balancing mode. The TSMC 0.18 μm high-voltage BCD CMOS process design kit, which offers 1.8V and 5V low-voltage transistors as well as 20V high-voltage LDMOS transistors, was used for this chip. It is assumed that 1.8V, 5V, 15V, and 20V supply voltages are available from the supply chip as specified in FIG. 2.

### Digital-to-Analog Converter

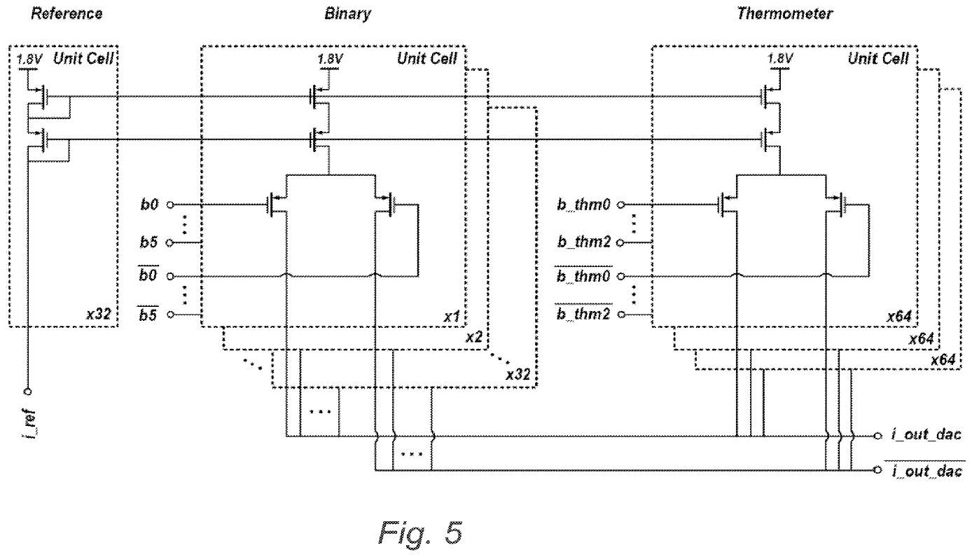

An 8-bit current-steering digital-to-analog converter (DAC) [3] is realized to generate the required arbitrary differential current stimulation signals. As shown in FIG. 5, control bits b₀-bs correspond to binary-weighted analog currents. Two additional control bits, b₆ and b₇, are subsequently decoded into control signals b_thmo₀-b_thm₂ that correspond to three thermometer-weighted analog currents. This configuration is used to minimize the differential nonlinearity. The reference current i_ref is set to 8 μA. The following equations give the expressions for output currents i_out_dac and i̅_̅o̅u̅t̅_̅d̅a̅c̅.

$$\\ \begin{matrix}i\_out \_dac = \frac{i\_ref}{32} [ {{\sum }^{5}}_{k = 0} {2}^{k} \cdot {b}_{k} + {{\sum }_{k = 0}}^{2} 64 \cdot {b\_thm}_{k} ] & ( 1 )\end{matrix}$$

$$\\ \begin{matrix}\overline{i\_out \_dac} = \frac{i\_ref}{32} [ {{\sum }^{5}}_{k = 0} {2}^{k} \cdot \overline{{b}_{k}} + {{\sum }_{k = 0}}^{2} 64 \cdot \overline{{b\_thm}_{k}} ] & ( 2 )\end{matrix}$$

The full-scale current available is 63.75 μA and the resolution is 250 nA. For each stimulation cycle, there are always 32 samples; thus, the clock frequency for this DAC is set to 4.8-640 KHz.

### Amplitude Control Circuit

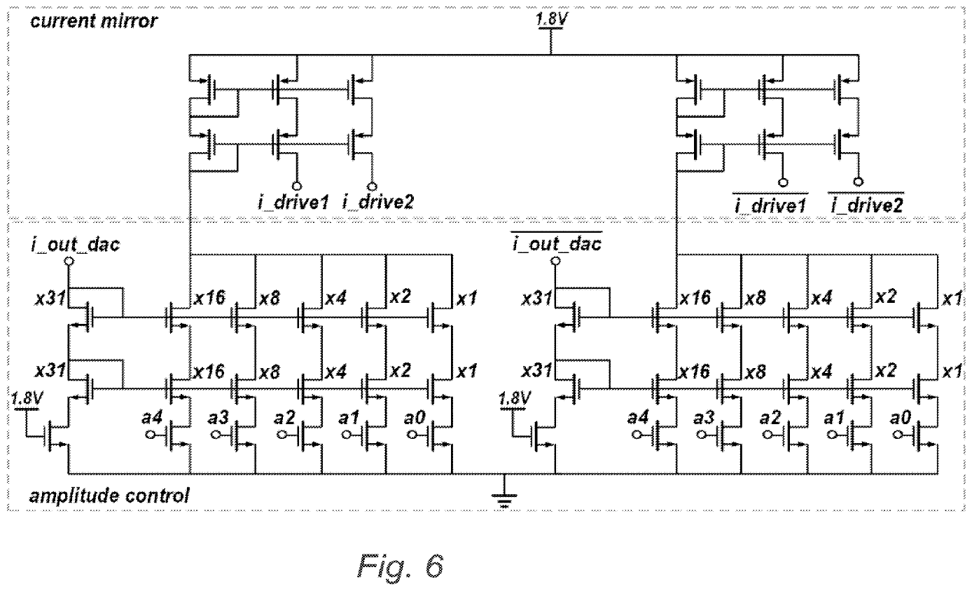

Since the DAC is designed to always operate with its full-scale amplitude, in order to generate differential current signals with tunable amplitudes, a 5-bit amplitude control block is designed as shown in FIG. 6. The output currents from the DAC, i_out_dac and i_out_dac, are fed into this block. The generated signals are then mirrored to i_drive1, i_drive2, t̅_̅d̅r̅i̅v̅e̅1̅, i̅_̅d̅r̅i̅v̅e̅2̅ and subsequently fed into the high-voltage drive & charge balance block. The following equations give the expressions for these signals.

$$\\ \begin{matrix}i\_drive 1 = i\_drive 2 = \frac{i\_out \_dac}{32} {{\sum }_{k = 0}}^{4} {2}^{k} \cdot {a}_{k} & ( 3 )\end{matrix}$$

$$\\ \begin{matrix}\overline{i\_drive 1} = \overline{i\_drive 2} = \frac{\overline{i\_out \_dac}}{32} {{\sum }_{k = 0}}^{4} {2}^{k} \cdot {a}_{k} & ( 4 )\end{matrix}$$

### High-Voltage Drive and Charge Balance Circuit

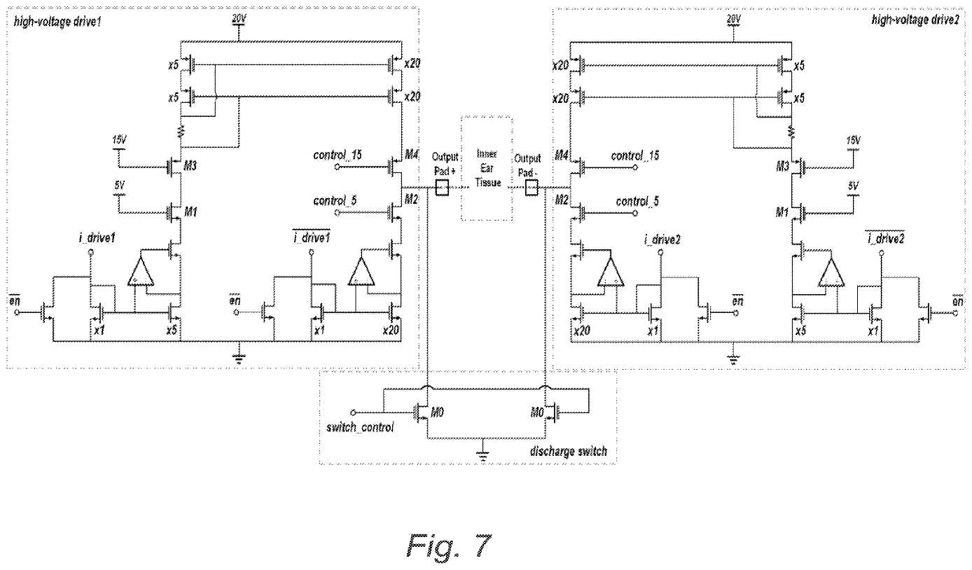

FIG. 7 shows the detailed schematic of the high-voltage drive & charge balance block. Input currents to i_drive1, i_drive2, i̅_̅d̅r̅i̅v̅e̅1̅, i̅_̅d̅r̅i̅v̅e̅2̅ are provided to this circuit from the outputs of the FIG. 6 amplitude control circuit. When e̅n̅ goes high the driver is in the charge balance mode. In this mode the outputs are set to a high-impedance state by setting control_5 to 0V and control_15 to 20V; at the same time switch_control is set to 5V so that transistors M0 pull the outputs close to ground, thereby discharging any residual charge inside the ear tissue. When e̅n̅ goes low the driver is in the stimulation mode, in which the biasing is turned on and transistors M0 are turned off.

In the FIG. 7 schematic a double-gate symbol corresponds to either a high-voltage transistor or a low-voltage transistor sitting inside a high-voltage well; a single-gate symbol corresponds to a standard 1.8V transistor. Transistors M1-M4 are 20V high-voltage LDMOS transistors; all other transistors with a double-gate symbol correspond to 1.8V transistors sitting inside high-voltage wells. The electrical rules for this process require that the maximum voltage magnitude between the gate and source of any high-voltage transistor not exceed 5V. LDMOS transistors M1-M4 are biased as shown to ensure that they do not break down in the presence of the high-voltage output swing. The op-amps are utilized in a feedback configuration to ensure precise current mirroring, and the dominant poles of the two feedback loops are set by the output loading of the op-amps. Note that this circuit amplifies the amplitude of the input current signal by a factor of 20. The various current mirror ratios in each driver are designed to maintain a low quiescent power dissipation, while maintaining good layout matching.

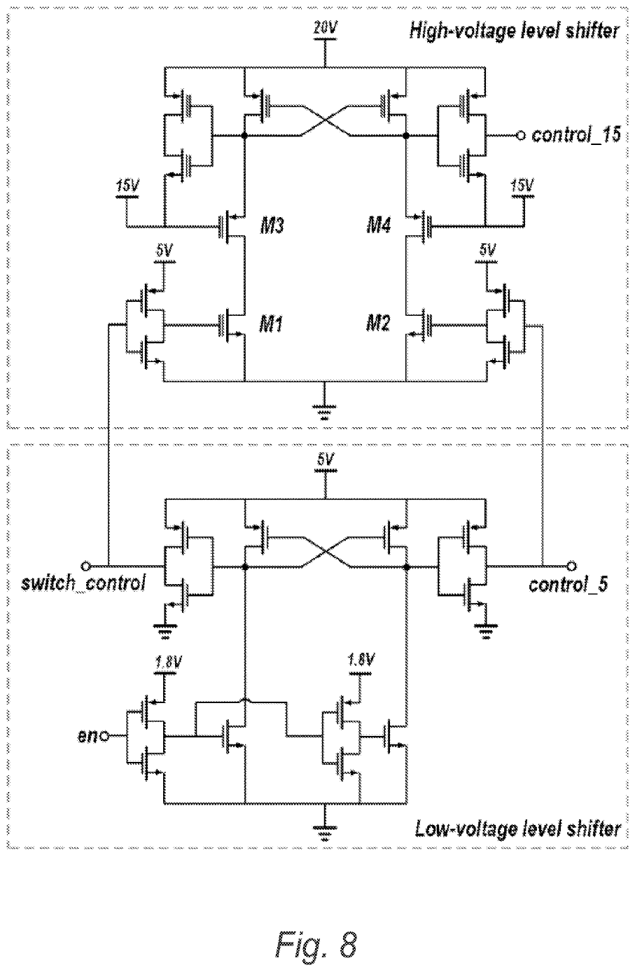

The digital control signals coming from the central control unit provide 0 and 1.8V logic levels while the bias voltage control_5 requires 5V (0V) for the driving (charge balance) phase; the bias voltage at control_15 requires 15V (20V) for the driving (charge balance) phase. Thus, level shifters are required as shown in FIG. 8. In this circuit the low-voltage level shifter functions to provide the 0 and 5V levels [4] needed to bias control_5. Note that the e̅n̅ signal in FIG. 7 is the negated e̅n̅ signal in FIG. 8. The high-voltage level shifter functions to convert the 0 and 5V levels to 20 and 15V levels, respectively, that are used to bias control_15. The gates of M3 and M4 are biased at 15V, which ensures the source-to-gate voltages of M3 and M4 will never exceed the maximum 5V break-down limit. All other transistors inside this high-voltage level shifter with double-gate symbol denote 5V low-voltage transistors sitting inside high-voltage wells; these low-voltage transistors allow for higher switching speeds and conduct lower leakage currents as compared to the high voltage transistors. The propagation delay of the high-voltage level shifter is less than 3.5 ns across PVT.

### Digital Circuit Blocks

The proposed core chip also contains a serial-to-parallel converter, central control unit, SRAM, and ROM as shown in FIG. 3. All these digital circuit blocks were built with the standard digital cells from TSMC 0.18 μm 1.8V standard digital library provided with the BCD process design kit. The functions of these digital circuit blocks are to ensure normal operation of the entire core chip as discussed in the system description part for all application scenarios.

### Simulation Results

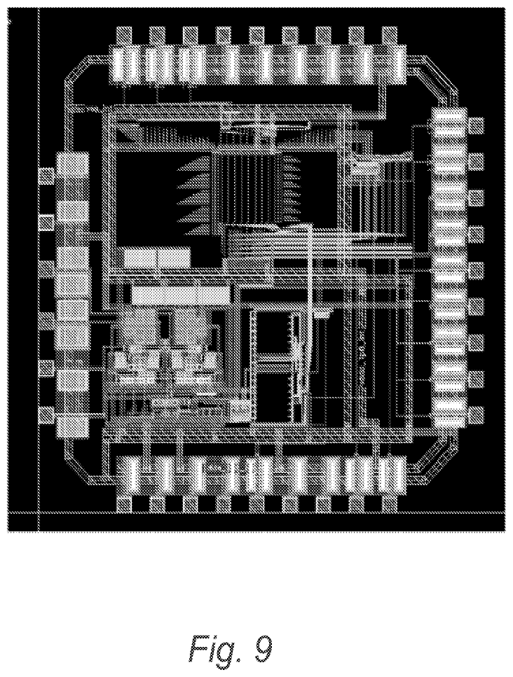

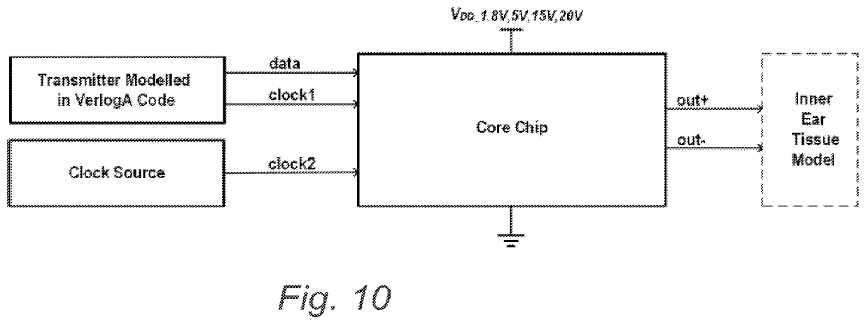

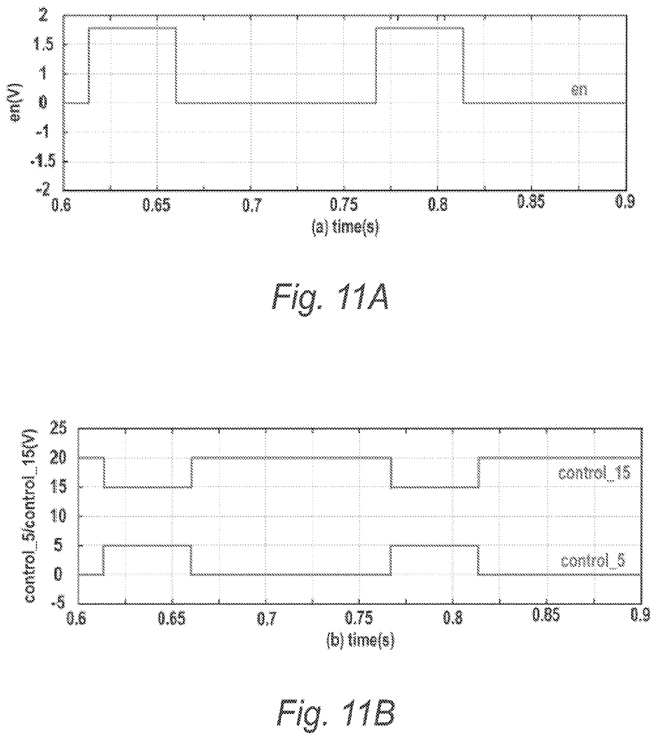

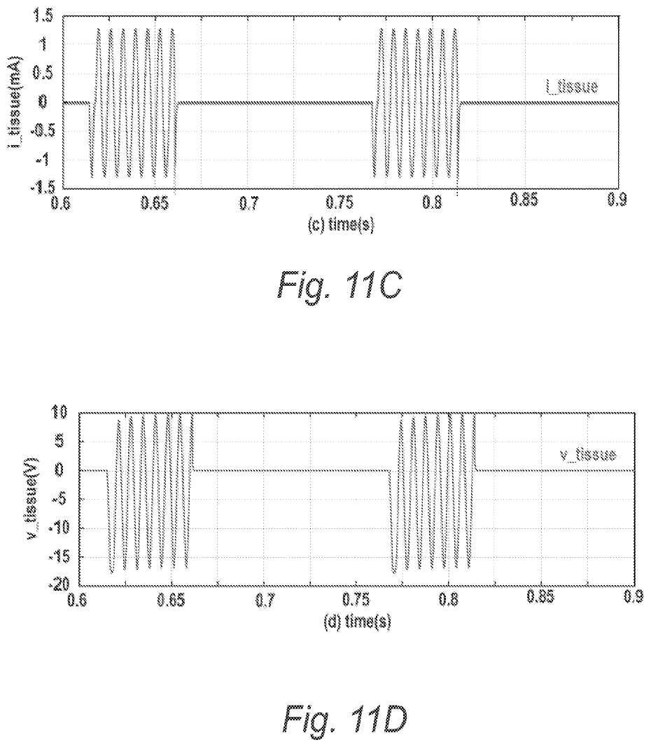

The core chip was simulated, including extracted parasitics from the layout, using the TSMC 0.18 μm BCD process design kit. The layout of this chip, with dimensions 2.34 mm×2.33 mm, is shown in FIG. 9. The test bench used to perform the top-level simulation is shown in FIG. 10. The simulated results are shown in FIG. 11. In this figure, the time-domain waveforms shown, from-top-to-bottom, are e̅n̅, control_15, control_5, the current flowing into the era tissue, the voltage across the ear tissue. The period of one stimulation cycle is 6.67 ms (corresponding to a 150 Hz stimulus frequency), the amplitude of the current stimulus reaches 1.25 mA, the repetition time is 155 ms and the duty cycle is about 30%.

### CONCLUSION

A core chip used in a tinnitus treatment has been presented. This chip is intended to be part of a miniature multi-chip module that will be implanted in a human ear for use as a tinnitus treatment. Post-layout simulations demonstrate that the chip output can provide a current stimulus waveform with 1.25 mA amplitude, with a variable charge balance phase to remove residual charge from the inner ear.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

M.-W. Suh et al., “Electric hearing and tinnitus suppression by noninvasive ear stimulation,” Hearing Resesarch, vol. 415, 1 Mar. 2022. doi: 10.1016/j.heares.2022.108431.
X.-H. Qian et al., “A bone-guided cochlear implant CMOS microsystem preserving acoustic hearing,” 2017 Symposium on VLSI Circuits, 2017, pp. C46-C47, doi: 10.23919/VLSIC.2017.8008542.
H. Pu, O. Malekzadeh-Arasteh, A. R. Danesh, Z. Nenadic, A. H. Do and P. Heydari, “A CMOS Dual-Mode Brain-Computer Interface Chipset With 2-mV Precision Time-Based Charge Balancing and Stimulation-Side Artifact Suppression,” IEEE Journal of Solid-State Circuits, doi: 10.1109/JSSC.2021.3108578.
Y. Kanno, H. Mizuno, K. Tanaka, and T. Watanave, “Level converters with high immunity to power-supply bouncing for high-speed sub-1-V LSIs,” Symp. on VLSI Cir., pp. 202-203, June 2000

What is claimed is:

1. A system for stimulating structures behind and proximate to a subject's ear, the system comprising:
a multi-chip module that is powered and controlled wirelessly, the multi-chip module having a size such that it is implantable in a subject's middle or inner ear, wherein the multi-chip module is configured to minimally-invasively electrically stimulate a structure in a subject's external ear, middle ear, and/or inner ear; and
transmitter configured to wirelessly (or connected via a wire) transmit control signals and power to the multi-chip module.

2. The system of claim 1, wherein the multi-chip module includes a first electrode configured to contact the structure in the subject's external ear, middle ear, and/or inner ear and a minimum of one other electrode configured to contact another proximate structure to operate as ground.

3. The system of claim 1, wherein the transmitter is configured to transmit an encoded control signal having information about waveform, frequency, and amplitude to be used for stimulating the structure in the subject's external, middle, and/or inner ear.

4. The system of claim 3, wherein the waveform is a periodic waveform.

5. The system of claim 3, wherein the structure in the subject's external ear, middle ear, and/or inner ear is stimulated with a signal having a voltage between 0 and 20 volts and/or a frequency from 20 Hz to 20 kHz and/or a current up to 1.25 mA.

6. The system of claim 1, wherein the structure in the subject's external ear, middle ear, and/or inner ear is a cranial nerve, a branch thereof, or a structure in close proximity to a cranial nerve.

7. The system of claim 1, wherein the multi-chip module is implanted in a subject to stimulate the facial nerve (cranial nerve VII) or one of its branches and is used to treat facial paralysis, facial spasms, synkinesis, obesity, or weight loss.

8. The system of claim 1, wherein the multi-chip module is implanted in a subject to stimulate the vestibulocochlear nerve (cranial nerve VIII) and is used to treat hearing loss, tinnitus, or balance issues.

9. The system of claim 1, wherein the multi-chip module is implanted in a subject to stimulate the glossopharyngeal nerve (cranial nerve IX).

10. The system of claim 9, where the multi-chip module is used to treat subject with salivation disorders or dry mouth.

11. The system of claim 1, wherein the multi-chip module is implanted in a subject to stimulate the vagus nerve (cranial nerve X).

12. The system of claim 1, wherein the multi-chip module is used to treat epilepsy, depression, anxiety, rheumatologic, metabolic, gastrointestinal, urologic, infectious, a neurological or other psychiatric disorder.

13. The system of claim 1, wherein the multi-chip module includes a core chip including:
a central control unit;
a digital-to-analog converter in electrical communication with the central control unit, with the digital-to-analog converter output determined by a first set of control bits corresponding to binary-weighted analog currents and a second set of control bits that are decoded into control signals corresponding to thermometer-weighted analog currents, the digital-to-analog converter outputting a first and second output current;
an amplitude control block driven by the digital-to-analog converter configured to receive the first and second output current from the digital-to-analog converter and to generate differential current signals with tunable amplitudes; and
a charge-balanced H-bridge stimulator in electrical communication with the amplitude control block, the charge-balanced H-bridge stimulator configured to receive the differential current signals with tunable amplitudes from the amplitude control block, the charge-balanced H-bridge stimulator configured to operate in a first mode in which residual charge inside ear tissue is discharged and a second mode in at least one cranial nerve is stimulated.

14. The system of claim 13, wherein the digital-to-analog converter is configured to operate with its full-scale amplitude.

15. The system of claim 13, wherein the core chip further includes static random-access memory (SRAM) that is driven by the central control unit, the SRAM being configured to store data that specifies signal parameters.

16. The system of claim 15, wherein the core chip further includes word selection circuitry in communication with the SRAM and the central control unit.

17. The system of claim 16, wherein the core chip further includes read-only memory (ROM) driven by the central control unit, the ROM being configured to store data that specifies signal parameters.

18. The system of claim 17, wherein the core chip further includes a serial-to-parallel converter configured to convert signal data into parallel data that is provided to the central control unit.

19. The system in claim 1, wherein the implanted device and non-implanted device have a unidirectional or bidirectional communication.

20. The system in claim 1, wherein the user has a handheld controller that allows changing parameters or a device applying artificial intelligence to change stimulation parameters based on physiological and non-physiological measurements.
